(19) United States
(12) Patent Application Publication
(10) Pub. No.: US 2003/0212383 A1
(43) Pub. Date: Nov. 13, 2003

(54) System and methods for reducing intraocular pressure

(76) Inventors: Dana Cote, Saugus, MA (US); Margaret Mulhern, Groton, MA (US); Robert Pierce, Wrentham, MA (US); Thaddeus Wandel, Croton, NY (US); Vladimir Stoy, Princeton, NJ (US)

Correspondence Address: David W Highet, Becton Dickinson & Company, 1 Becton Drive, Franklin Lakes, NJ, 07417-1880, US

(21) Appl. No.: 10/182,833

(22) PCT Filed: Jan. 5, 2001
(86) PCT No.: PCT/US01/00350

(51) Int. Cl. A61M 25/00; B32B 1/06; B32B 1/08; B32B 31/04

(52) U.S. Cl. 604/523; 264/171.12

(57) ABSTRACT

The present invention provides systems and methods for reducing intraocular pressure, thereby to treat glaucoma and other disorders. The systems of the present invention include a shunt insertable across the clear cornea and a delivery device for inserting the shunt in the transcorneal position. The shunt has a body with a head at one end and a foot at the opposite end, and a channel therethrough permitting the passage of aqueous humor from the anterior chamber to the external surface of the cornea. A removable filter is positioned within the channel to regulate aqueous humor outflow and to resist the incursion of microorganisms.

### RELATED APPLICATION

The present application is related to and claims benefit of U.S. Provisional patent application 60/175,658, “Glaucoma Pressure Relief Valve and Drug Delivery Device,” filed Jan. 12, 2000, the contents of which are hereby incorporated by reference.

### BACKGROUND

1. Field of the Invention

The invention relates generally to systems and methods for reducing intraocular pressure. In one embodiment, the invention relates to implantable devices for drainage of aqueous humor to relieve high intraocular pressures characteristic of glaucoma.

2. Description of Related Art

The eyeball is a substantially spherical structure whose shape and tone is maintained by endogenous fluid materials that fill an external hollow collagenous globe. The interior of the eyeball is divided into two chambers, the anterior chamber and the posterior chamber. Suspended between these chambers are the ocular lens and its supporting and related tissues. The posterior chamber is filled with a gelatinous material called vitreous humor that is not thought to contribute significantly to the pressure level within the eyeball, termed intraocular pressure (IOP). In contrast, the anterior chamber is filled with a watery fluid called aqueous humor that is constantly being produced and resorbed. This fluid exerts pressure against the overlying cornea and against all structures surrounding it. If the amount of aqueous humor produced is excessive, pressure within the anterior chamber and within the eyeball will rise. Normal IOP results from a healthy equilibrium between production and resorption of aqueous humor.

Aqueous humor is produced behind the base of the iris and flows into the anterior chamber. Resorption takes place through the trabecular meshwork system, from whence the fluid passes into scleral vessels to be taken up into the bloodstream. A certain range of pressures in the anterior chamber is considered normal, generally between 10 and 21 mm Hg. The pressure within the anterior chamber is determined by how rapidly aqueous humor is produced and how rapidly it is drained through the trabecular meshwork system. Obstruction to the drainage system may be a cause of elevated intraocular pressure. Persistence of elevated IOP produces the condition known as glaucoma, wherein an elevated IOP may damage the optic nerve and affect vision, leading eventually to blindness if not properly treated.

A variety of treatments for glaucoma are available. Medical therapies endeavor to reduce IOP improving fluid outflow or reducing fluid production. Available medical treatments may include topical ophthalmic or systemic medications. Medical management may fail, however, because of poor patient compliance, high cost, or any one of a number of well-recognized complications and side effects. In the event that medical management is unsuccessful, more invasive treatments can be offered to the patient either to alter the normal anatomy or to introduce implantable drainage devices for relieving excesses of aqueous humor. For example, laser surgery may be recommended to alter the anatomy of the trabecular meshwork and enhance anterior chamber drainage; other laser-mediated ophthalmological procedures are also available for glaucoma treatment. Glaucomatous eyes that continue to have elevated intraocular pressures despite medical treatment and laser intervention may require a definitive surgical procedure.

As one example, a conventional type of surgical intervention aims to create a fistula or other drainage channel out of the anterior chamber of the eye. The aqueous humor is thereby directed to flow into a surgically created subconjunctival or scleral pocket, often called a “bleb,” from whence the fluid can be reabsorbed into the bloodstream. This operation reduces intraocular pressure by allowing excess fluid to flow out of the anterior chamber. Several known limitations accompany such procedures, however. First, normal wound healing tends to interfere with the patency of the fistula and with the dimension of the drainage pocket, so that these operations may have an unacceptable rate of failure. To increase the success rate of this type of surgery, physicians may recommend adjuvant treatment with agents that modulate normal wound healing. Such treatment increases the incidence of a second sort of problem associated with these procedures: excessive or overly rapid outflow of aqueous humor. It is well known that removal of too much aqueous humor too quickly can reduce intraocular pressure precipitously to dangerously low levels, a condition called hypotony, potentially causing a number of sight threatening complications. To prevent this problem, the surgical site must heal sufficiently well to produce controlled aqueous humor drainage. For this to occur, normal wound healing is essential. Those treatments that inhibit wound healing therefore increase the risks associated with excessive aqueous humor drainage. A third kind of problem accompanies this type of conventional drainage procedure: an increased risk of infection. Drainage of aqueous humor into a scleral or subconjunctival bleb poses a risk for infection by providing a fluid milieu that microorganisms can invade. Furthermore, if an infection becomes established in the fluid-filled pocket, the microorganisms can travel retrograde through the drainage channel to enter the anterior chamber of the eye and infect it as well, a much more serious condition.

To address some of the problems associated with conventional surgery, a number of implantable devices have been proposed that endeavor to drain excessive fluid from the anterior chamber. The problems described above that affect soft tissue surgery also affect implantation surgery, however. Wound healing mechanisms are still called into play, even though the surgery includes the installation of an intraocular implant. Indeed, artificial materials may overstimulate local wound healing, leading to excessive scar tissue formation. Furthermore, controlling the outflow rate of aqueous humor remains essential, even if an artificial device is involved in the process. In addition, infection remains a risk. With a mechanical conduit available to transmit microorganisms from the outside to the interior of the eye, some mechanism is desirable for discouraging retrograde infection. Finally, the eye, like most tissues of the body, has limited tolerance for the long-standing presence of artificial materials. A locally positioned implant may irritate the surrounding tissues. The eye, of course, is particularly sensitive. A device to be implanted on the surface of the eye may be perceived by the patient as a chronic, persistent and bothersome foreign body. Finally, since eye tissues are so delicate, implants must be designed and placed so that they do not damage vulnerable adjacent, subjacent or overlying tissues. Even if properly positioned initially, however, the implant can be displaced by local tissue motion or can be extruded by constrictive wound healing processes.

A variety of devices in the prior art purport to provide solutions for some or all of these problems. For example, certain prior art devices shunt aqueous humor to a reservoir or drainage area that is implanted in the sclera or subconjunctivally. As mentioned earlier, however, these devices face the problems of regulating aqueous outflow, resisting infection and avoiding local tissue irritation and trauma. The first problem, regulating aqueous outflow, arises because the drainage rate of this fluid depends substantially on the mechanical characteristics of the implant until there has been sufficient wound healing to restrict fluid outflow biologically. Effective balancing of biological and mechanical resistance to aqueous humor outflow remains a problem for implant-based drainage procedures. Prior art devices utilize a variety of mechanisms to restrict aqueous outflow. Each of these mechanisms, though, may become a liability once wound healing has been established. Restrictive elements within the implant, when combined with the restriction effected by wound healing, may inordinately reduce the rate of aqueous humor outflow, possibly to non-therapeutic levels. The second problem, the possibility of intraocular infection, arises because the presence of an implant provides a conduit through which bacteria can gain entry to the interior of the anterior chamber. Certain rior art drainage devices have introduced filters or valves or other conduit systems to impede the retrograde transmission of infection into the anterior chamber. These mechanisms have limitations, however: even when effective in resisting the transit of microorganisms, they have hydraulic effects on fluid outflow that may also impair effective drainage. Finally, the problem of local tissue tolerance arises with certain prior art devices because these foreign bodies may incite tissue reactions culminating in local inflammation or extrusion, and may further be perceptible or uncomfortable for the patient: these reactions to the presence of the implant may make its use clinically unsuitable.

Devices placed through the clear cornea to effect aqueous humor drainage are intended to avoid certain limitations accompanying scleral or subconjunctivally implantation. Certain devices, for example U.S. Pat. No. 3,788,327 and U.S. Pat. No. 5,807,302, and U.S. Pat. No. 5,743,868, provide for transcorneal conduits that drain anterior chamber fluid onto the surface of the cornea to mix with the tear film. The devices taught in the abovementioned patents contain certain features directed to the problems of outflow regulation, microorganism restriction, local tissue compatibility, and positional stability. These problems, as previously discussed, affect transcorneal devices as well. There remains a need, therefore, for a biocompatible anterior chamber drainage device that permits the well-controlled outflow of aqueous humor despite vagaries of wound healing. There remains a further need for a drainage device that can limit the ingress of microorganisms and thereby protect the interior of the eye from infection. In addition, there remains a need for an ophthalmological drainage device that is well tolerated and comfortable for the patient. Finally, the problem of positional stability has not been solved satisfactorily. A need exists in the art for a drainage device that can be securely and reliably positioned without fear of dislodging, migration, or extrusion.

In addition to the aforesaid needs for permanent or durable drainage of the anterior chamber in conditions such as glaucoma, there are additional needs for temporary anterior chamber drainage or decompression. For example, IOP elevation over short intervals (1 hr -2 wks) may exist following a number of ophthalmological procedures, including cataract extractions and repair of retinal detachment. Moreover, a physician may find it advantageous to use a shunt to temporarily control IOP in glaucoma before embarking upon other surgical procedures for the disorder that do not employ long-term shunting. A need exists for a device to fulfill the need for short-term anterior chamber drainage in these and similar situations.

A further need exists for providing a delivery system specifically adapted for atraumatic insertion of a transcorneal drainage device. Advantageously, such a delivery system would be able to hold the drainage device securely so that it could be positioned by the surgeon. Such a delivery system would further permit the ready release of the drainage device when it is to be inserted through the cornea. It is further desirable that the delivery system be fabricated to avoid introducing any additional damage to the delicate tissues of the corneal epithelium and stroma.

### SUMMARY OF THE INVENTION

It is an object of the present invention to provide systems for reducing intraocular pressure. The systems of the present invention may include a shunt insertable through the clear cornea of the eye into the anterior chamber to drain aqueous humor therefrom. The shunt may include a substantially cylindrical body with a channel through with that permits drainage of aqueous humor from the anterior chamber to the external surface of the clear cornea; the shunt may further include a head that rests against the outer surface of the clear cornea, a foot that rests against the inner surface of the cornea and an elongate filter retainable within the channel of the body that regulates the flow rate of aqueous humor therethrough and that minimizes the ingress of microorganisms. In one embodiment, aqueous humor is able to flow through an aperture in the foot to enter the channel in the body and pass therethrough, to exit through a slit in the head, flowing onto the surface of the cornea. In one embodiment, the head and the foot are formed integrally with the body. In another embodiment, the head, the foot, or the body may be made from a dehydratable polymer. In certain embodiments, the external surface of the head or of the foot may be configured to minimize cellular adhesion or adherence. In certain embodiments, the external surface of the body may be configured to encourage tissue adhesion or adherence, or to be attractive. The foot may be specifically shaped to facilitate introduction of the shunt through the cornea. In certain embodiments, the body is smaller in circumference than the head or the foot. The elongate filter may be retained within the channel of the body by impaction or by any other appropriate mechanism. The elongate filter may be positioned at the proximal end of the body or in any other position therein.

In other embodiments, the systems of the present invention may include an implant that can be placed across the cornea to drain the anterior chamber of the eye. The implant may include a head, a foot, a tubular conduit between the foot and the head that has an interior channel in fluid communication with the anterior chamber, and a filter that can be impacted within the anterior chamber to regulate outflow of aqueous humor and to restrict incursion or minimize ingress of microorganisms or obstruct their passage.

In yet other embodiments, the systems of the present invention may include a transcorneal shunt and may further include a delivery device for implanting the shunt in this transcorneal position. In certain embodiments, the transcorneal shunt to be implanted with the delivery device may have a head, a foot, a substantially cylindrical body between the head and the foot having a channel therethrough, and a filter positioned within the channel to regulate the flow rate of aqueous humor through the channel and further to restrict the ingress of microorganisms. In certain embodiments, the delivery device may include a tip dimensionally adapted for holding the shunt and for positioning the shunt for insertion through the external surface of the cornea, and may further include a plunger slidable from a proximal position to a distal position wherein sliding the plunger dislodges the shunt and urges it through the external surface of the cornea into a transcorneal position.

It is a further object of the present invention to provide methods for decreasing anterior chamber fluid pressure, thereby to treat glaucoma and other disorders characterized by elevated anterior chamber pressure. These methods may include the steps of providing a transcorneal shunt, providing a delivery device for positioning the shunt in the transcorneal position, incising a pilot hole through the exterior surface of the cornea to permit the insertion of the shunt therethrough, and employing the delivery device to insert the shunt into the transcorneal position. In one practice of the invention, the shunt that is provided may have a substantially cylindrical body, the head, a foot and a filter. It is yet another object of the present invention to provides methods for temporary drainage of anterior chamber fluid, thereby to decrease intraocular pressure. Temporary drainage is understood to take place over a short term, for example, from one hour to several weeks, using a device that may be removable at the conclusion of the temporary drainage period or that may be biodegradable, to be resorbed at the end of that temporary period. Such a device may be useful for implantation following those procedures that might be followed by increases in IOP, or may be useful as a temporary correction for disorders characterized by increased IOP.

The shunt according to the present invention is intended to solve certain of the abovementioned problems that have persisted within the ophthalmological arts for treatment of elevated IOP. First, the shunt, its delivery device and the methods for their use are adapted for positioning of a drainage system across the clear cornea, thereby avoiding the difficulties that accompany subconjunctival or subscleral drainage. Second, the outflow of aqueous humor is consistently regulated by a filtration system without implicating mechanisms of wound healing, so that a predictable outflow rate can be calculated to avoid the dangers of hypotony on one hand and inadequate drainage on the other. Third, the filter provides a tortuous path to inhibit bacterial ingress; in addition, the slit opening in the head is shaped and sized to resist bacterial invasion; furthermore, the head itself is fabricated from a material that resists cellular adhesion, including the adhesion of microorganisms. Fourth, the device is made of materials well tolerated by the cornea. The head and the foot resist cellular adhesion and discourage scarring over the device, while the body is made of materials that encourage cellular adhesion, thereby to affix the device securely in the transcorneal position. These and other objects, features and advantages of the present invention will become more evident from the following discussion and drawings, wherein like numbers represent like components.

### BRIEF DESCRIPTION OF THE DRAWINGS

FIG. 1 is a perspective drawing of an embodiment of the present invention.

FIG. 2 is an exploded view of an embodiment of the present invention showing an insertion path of the filter.

FIG. 3 is a cross-section view of an embodiment of the present invention.

FIG. 4 is an anatomic cross section showing a shunt in position according to the present invention.

FIG. 5 is a schematic diagram of an embodiment of the present invention.

FIGS. 6A-D show perspective and cross-sectional views of a delivery device according to the present invention.

FIGS. 7A-B show a perspective and a cross-sectional view of an alternative embodiment of a delivery device according to the present invention.

### DETAILED DESCRIPTION

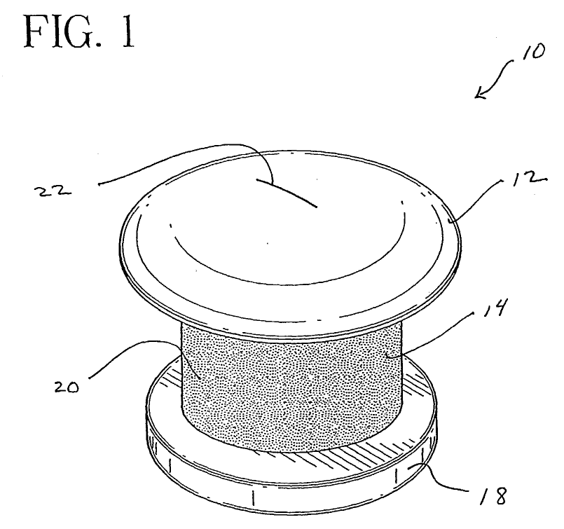

With reference to FIG. 1, a perspective view of a shunt 10 according to the present invention may be seen. In a representative embodiment, the shunt 10 may be approximately one millimeter long with an outer diameter of approximately 0.5 mm. While the shunt 10 illustrated in this and the following figures is shown as a cylindrical structure, it is understood that other shapes of tubular conduits may be suitable as well. For example, the shunt 10 may assume a more oval shape or a more lenticular shape. FIG. 1 shows the shunt 10 from its top or external aspect. The shunt 10 dimensionally adapted for transcorneal positioning. The head 12 will be located on the external or epithelial surface of the cornea when the shunt 10 is in position. As shown in this figure, the head 12 may be dome-shaped to provide a continuous transition surface from the device to the cornea. This shape may also be well tolerated by the patient's eyelid. While this shape seems particularly advantageous, other shapes of the head may be designed to provide the same advantages. For example, a minimally protruding flat head 12 with rounded edges may be equally well tolerated. Other appropriate designs may be determined using no more than routine experimentation. The undersurface (not shown) of the head 12 may be flat or curved suitably to match the shape of the corneal surface whereupon the device is to be positioned. The head 12, the body 14, and the foot 18 may all be formed integrally as a unit, or the head 12 or the foot 18 may be formed integrally with the body. In another embodiment, each component may be disassemblable from the others.

Copolymers of hydroxyethyl methacrylate (HEMA) may be used in the fabrication of components of the shunt. In one embodiment, the head 12 is formed from a smooth material to inhibit tissue and bacterial adherence and is highly hydrated and wettable with tears. The head 12 may have a surface ingredient comprising a HEMA polymer such as IHEMA plus methacrylic acid that is well known in the art for inhibiting cell adhesion. As an example, poly 2-hydroxyethyl methacrylate (PHEMA) may be used for the shunt casing. In one embodiment, the base material for the tissue integration layer coating that attracts cells may include HEMA and cyclohexylmethacrylate. Covalently crosslinked hydrogels used in contact lenses and having equillibrium water content at least 15% by weight (and more preferably at least 20% by weight), may be included in the composition of the casing, in particular copolymers of esters of acrylic and methacrylic acid with di- and polyhydroxy compounds. Examples of suitable polyhydroxy compounds include ethylenglycol, diethylenglycol, triethylenglycol, 1,2-propandiol, glycerol, glycerolmonoacetate, glucose and the like. Such esters may be further copolymerized with vinylpyrrolidone, acrylic and methacrylic acid, acrylamide, N-substituted acrylamide, and many other similar compositions, as will be apparent to practitioners in the art. A number of specific compositions of such hydrogels are known in the art, many of which would be suitable and readily,identifiable to skilled artisans using no more than routine experimentation. Typical crosslinkers are diacrylates and dimethacrylates of the above diols and polyols. In certain embodiments, the surface of the body 14 may include a tissue integration layer comprising a crosslinked polymer, for example a composition comprising HEMA and a alkylmethacrylate, particularly cyclohexylmethacrylate and particularly in such a composition where the said alkylmethacrylate is used in a higher concentration than HEMA. The tissue integration layer may be smooth, patterned or porous. In an exemplary embodiment, a shunt consistent with the present invention would be characterized by certain physical characteristics, including reversible hydration, shape memory, localized surface regions with hydrophilic or hydrophobic properties, localized surfaces with different hydration properties and localized surfaces having different cellular adhesion properties.

Bacterial invasion is further resisted by the slit 22 traversing the head 12. The slit 22 permits the outflow of aqueous humor that has passed through the shunt to flow onto the clear cornea, thereby to enter the tear film. While the slit 22 depicted in this figure is a single elongate aperture, it is understood that other slit configurations may advantageously provide for aqueous humor outflow and restriction of bacterial incursion. For example, a pattern of multiple small slits may be designed. Or, for example, a slit or series of slits may less elongated and more rounded than this figure depicts. Other slit arrangements may be readily envisioned by practitioners of ordinary skill.

The foot 18 may be made from materials similar to the head 12. This figure shows a top or outer surface of the foot 18 adapted for contact with the inner or endothelial surface of the cornea. As shown here, the foot 18 may be flat, or it may be curved to fit the shape of the corneal surface it contacts. Furthermore, the foot 18 may be tapered or frustoconical to facilitate its insertion through the cornea. In the depicted embodiment, the foot 18 is wider than the body 14. The inner surface (not shown) of the foot 18 bears an aperture through which aqueous humor enters the shunt 10. These and other features of the foot 18 will be shown in other figures.

With further reference to FIG. 1, the body 14 of the shunt 10 is positioned between and is connected to the head 12 and the foot 18. The body 18 may be made from a solid HEMA polymer and coated with a hydrogel, such as a copolymer of HEMA and cyclohexylmethacrylate, that serves to promote cell adhesion. The coating 20 of the body 18 is receptive to tissue attachment, so that the body 18 may be securely anchored in position. This feature enables the shunt 10 to resist in situ motion and displacement. Furthermore, this feature serves to prevent bacterial ingrowth along the transcorneal channel within which the shunt 10 is positioned. To further promote tissue ingrowth and cell attachment, the coating 20 of the body 18 may be treated with surface alterations such as texturing, roughening or introduction of patterned irregularities. Combining HEMA polymers that promote cell adhesion on the body 14 with HEMA polymers that resist cell adhesion on the head 12 and the foot 18 permits the shunt 10 both to become firmly attached to the cornea where the body 14 passes therethrough, and also to resist the attachment of bacteria to the head 12 with potential subsequent invasion.

It is understood in the art that devices made of HEMA are well tolerated by the eye. In addition, a device made from dehydrated polymer, such as HEMA, may be dehydrated to be reduced to a smaller size for implantation through a small incision. This feature may facilitate insertion of the shunt through a pilot hole or similar small access route with minimal tissue disruption. After a dehydrated shunt 10 according to the present invention is properly positioned, it may imbibe water from the surrounding tissues and swell to its predetermined size. Varying degrees of dehydration are possible, depending on the particular hydrogel formulation. Even if dehydration only yields a small decrease in size, this may facilitate implantation. Furthermore, implanting the dehydrated device in its transcorneal position and allowing it to imbibe water and hence enlarge will secure its tight fit in the intended position.

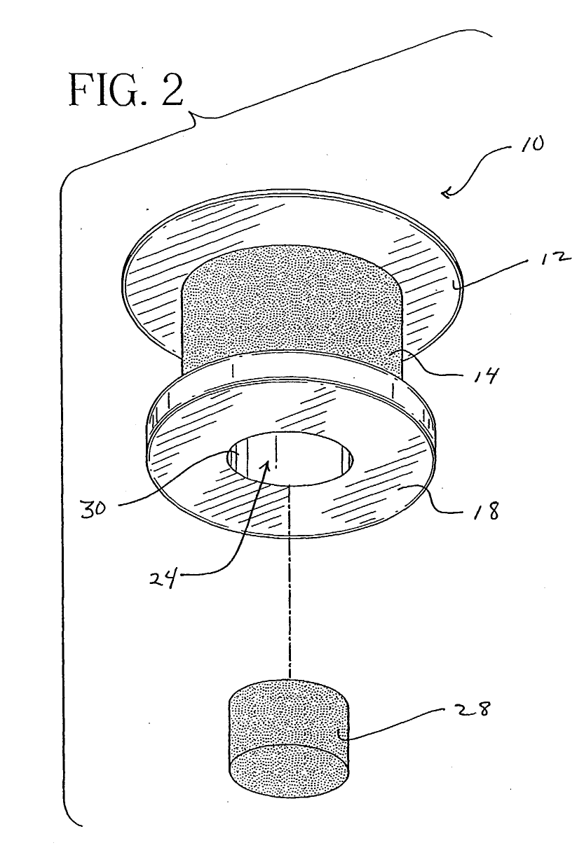

FIG. 2 presents a perspective view of the shunt 10 as seen from the bottom or interior aspect. In the depicted embodiment, when the shunt 10 is positioned anatomically, the foot 18 lies on the inner aspect or endothelium of the cornea and projects into the anterior chamber. In this figure, the body 14 and the head 12 may be also seen. The shunt 10 is provided with a channel 24 the passes through the foot 18 and the body 14 to approach the undersigned of the head. As illustrated in the previous figure, a slit (not shown) on the head 12 permits the egress of aqueous humor that has flowed through the channel 24. A filter 28 regulates the flow of aqueous humor from the anterior chamber to the external aspect of the eye and provides a tortuous path through the channel 24 to impede the passage of bacteria. In one embodiment, the filter 28 may be made of titanium. Other materials such as ceramics and polymers may also be suitable for the filter 28. In certain embodiments, the filter 28 is impactable within the channel 24 of the body 14. The filter 28 may be intended to form a permanent element of the shunt 10. Alternatively, the filter 28 may be removable and replaceable in those embodiments where access to the channel 24 is provided without disrupting the transcorneal position of the shunt 10. For example, a removable head 12 may permit access to the filter 28 so that it can be removed and replaced. As another example, the head 12 may be provided with an access port (not shown) located so that access to the filter 28 would be available without disrupting the position of the head 12. That access port and its attachment to the head 12 could, in certain embodiments, be integrated with the slit system described previously. Other arrangements may be readily envisioned by practitioners in these arts. The filter may be housed within a rigid housing. This housing may be inserted and removed from the shunt body 14 after the tissue integration layer has affixed the body 14 in position, without disrupting the affixation of the casing in the eye.

As shown in FIG. 2, the filter 28 may be fabricated as a cylinder to be inserted within the channel 24 by a press fit. In the illustrated embodiment, the channel 24 has smooth walls 30. The filter 28, with representative dimensions of approximately 0.02 by 0.02 in., abuts the wall of the channel 24 to be securely fixed therein. The depicted filter 28 contains a network of pores with pore size approximately 0.5 microns. The size of the pores is dimensionally adapted for controlling fluid flow rate at approximately two microliters per minute. This flow rate, obtained by fabricating the size of the pores and the length of the flow path to provide appropriate resistance to flow, is sufficient to reduce the excess intraocular pressure associated with glaucoma while preventing ocular hypotony. While the previously described arrangement of pore size and flow path length appears particularly advantageous for the systems of the present invention, it is understood that other arrangements of pore size and flow path length may also be suitable. It is further understood that hydraulic characteristics of metals, ceramics or polymers may vary and that specifications for filters made from these substances may vary also while still falling within the scope of the invention, with the intent of any filter being to provide consistent, predictable and pathophysiologically desirable rates of aqueous humor outflow while interfering with retrograde passage of microorganisms.

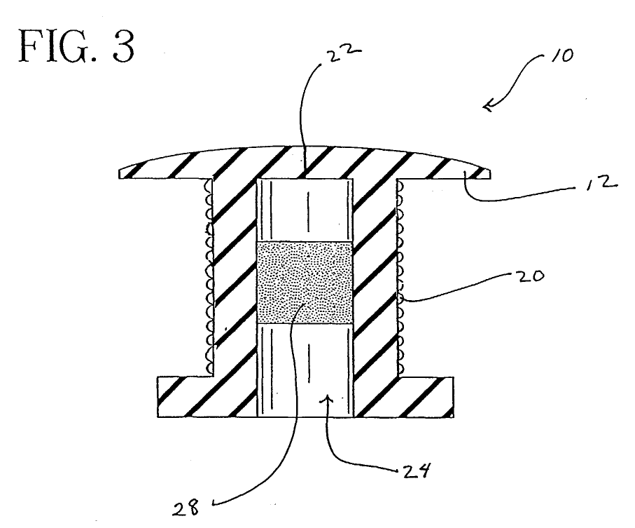

FIG. 3 shows a shunt 10 according to the present invention in cross-section. This figure illustrates a fluid path for aqueous humor from the anterior chamber through the channel 24 passing through the body 14 to drain out through the slit 22 in the head 12. This figure shows the head 12, the body 14 and the foot 18 all fabricated integrally as a unit. This figure also shows a single linear slit 22 penetrating the head 12. The depicted slit 22 extends axially through the head 12. Other slit arrangements may be envisioned as well. An irregular slit path, for example, may be provided. Multiple slits or a combination of slits and other shaped perforations may also be provided. In this figure, a coating 20 with an irregular surface has been applied to the outer aspect of the body 14. A filter 28 is shown disposed securely within the channel 24. As illustrated in this figure, the filter 28 occupies the mid portion of the channel 24. Other positions of the filter 28 may also be suitable. For example, the filter 28 may be positioned more proximally or more distally then is illustrated here.

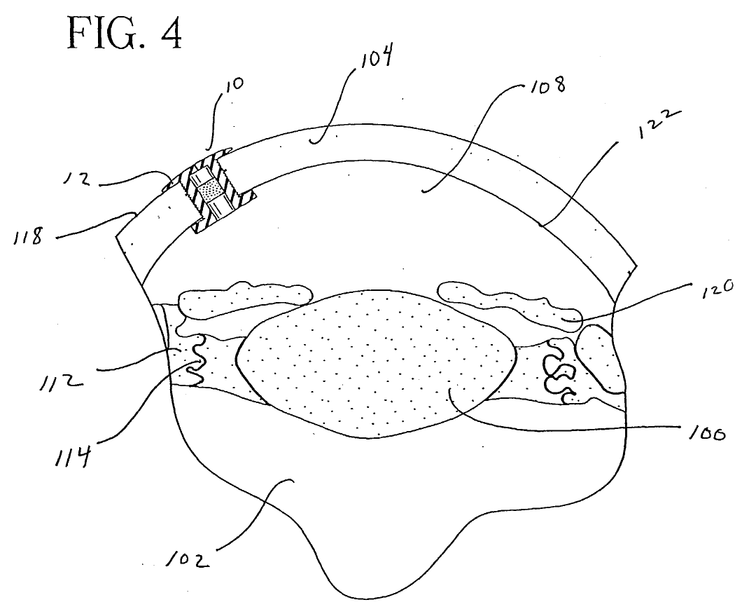

FIG. 4 shows an anatomic cross section with the shunt 10 in its anatomic position traversing the cornea 104. As previously described, surfaces of the depicted embodiment may be made from different materials with different properties, in particular, with a surface resistant to cell adhesion or protein deposition and with a surface attractive to cell adhesion, as described above. The head 12 of the device is seen resting on the corneal surface 118. The shunt 10 is provided with a passage therethrough that permits fluid within the anterior chamber 108 to flow across the clear cornea 104 to the outside surface of the eye. Fluid entering the interior passage of the shunt 10 will then exit the device and flow onto the outer corneal surface 118, from whence it commingles with the tear film. This figure shows the head 12 of the shunt 10 in contact with the outer corneal surface 118. This figure further shows the foot 18 in contact with the inner corneal surface 122, although such contact is not necessary for satisfactory positioning. In a representative positioning, the shunt 10 of the present invention may be placed in the superior aspect of the clear cornea, overlain by the upper lid during neutral gaze. Embodiments of the shunt 10 according to the present invention may be constructed to span the corneal stroma between the tear film on the outer corneal surface 118 and the anterior chamber 108. In certain embodiments, a shunt 10 may include at least the following components: (a) a body 14 made from a hydrogel and having an outer surface in direct contact with stromal tissue; (b) a head 12 protruding from the cornea and having an external surface in contact with the tear film and in at least intermittent contact with the inner aspect of the eyelid (not shown); (e) a foot 18 protruding into the anterior chamber 108. In the described embodiment, at least the external surface of the body 14 and the head 12 have different properties with respect to cell adhesion and water wettability. In a particularly preferred embodiment, the external surface of the head 12 is non-adherent for cells and is well wettable with tears and is highly hydrated, whereas the external surface of the body 14 is less hydrated and highly adherent for cells. FIG. 4 also schematically shows other anatomic structures. The lens 100 is shown dividing the anterior chamber 108 from the posterior chamber 102. Lateral to the lens 100 are the ciliary processes 114 of the ciliary body 112, which structures are responsible for the production of aqueous humor. Anterior to the lens 100 is the iris 120.

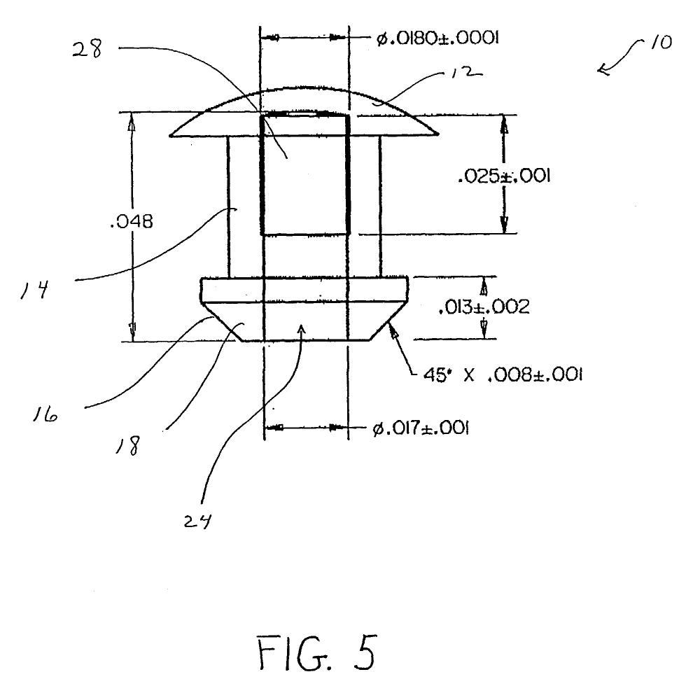

FIG. 5 illustrates schematically an embodiment of the shunt 10 according to the present invention. In the depicted embodiment, the body 14 is traversed by a channel 24 approximately 0.017 in. to 0.018 in. in diameter. In the depicted embodiment, the channel 24 is approximately 0.048 in. in length. A filter 28 is shown within the channel 24. The filter 28 has a vertical height of approximately 0.020 inches. It is advantageous that the filter be configured to retain microorganisms such as bacteria, viruses, fungi and spores thereof. The foot 18 is shown to have a tapered edge 16 to facilitate inserting the shunt 10 across the cornea. The tapered edge 16 depicted in this figure slants at a 45 degree angle over a distance of approximately 0.008 inches. The foot 18 may have an overall vertical height of approximately 0.013 inches. Other sizes and shapes of the foot 18 may be envisioned that facilitate insertion of the shunt 10 across the cornea while allowing the foot 18 to remain properly located within the anterior chamber. For example, the foot 18 may be provided with a folding or pleating arrangement which minimizes its size with dehydration and expands to a larger size with rehydration. In other embodiments, the foot 18 may have a frustoconical shape or an inverted frustoconical shape that can be folded to facilitate its insertion. In certain embodiments, the foot 18 is larger than the body 14, as is shown in this figure. While the filter 28 shown in this figure is positioned in distal end of the channel 24, other positions for the filter 28 are consistent with the present invention. For example, the filter 28 may be positioned more approximately in the channel 24, or it may occupy a made positioned in the channel, or it may be fabricated with pore size and fluid pathway length sufficient to allow the filter 28 to occupy substantially all of the channel 24.

In certain embodiments, a shunt 10 according to the present invention may be formed from a shape memory polymer that can be converted into a deformed shape suitable for insertion through a small incision, to return to its preselected shape in response to hydration or in response to body temperature. For example, a shunt 10 in the state of partial dehydration with a softening temperature T, that is higher than room temperature and preferably near body temperature may be initially inserted into the transcorneal position through an access incision (e.g., a slit, an excision, a puncture or any other access incision familiar to skilled artisans), and may then, upon rehydration and temperature increase, expand to assume its preselected size and shape.

Methods for manufacturing a shunt according to the present invention may include fabrication in a disposable mold or by machining with the tissue integration layer being applied as a curable composition. For example, the corneal implant or shunt can be cast from a mixture of HEMA, methacrylic acid, dimethacrylate crosslinker, and a free radical initiator in a single part silicone mold with a cavity formed by imprinting with a die shaped in a preselected shape. Alternatively, the corneal implant or shunt can be machined and then a tissue integration layer can be applied to an outer surface of the shunt. The tissue integration layer being a curable composition comprising a copolymer of HEMA with alkylmethacrylate, monomer HEMA, a dimethacrylate crosslinker, a free radical initiator and a volatile solvent. Other methods for manufacturing a corneal implant or shunt according to these systems and methods should be readily identifiable by practitioners of ordinary skill in the relevant arts.

Systems and methods of the present invention may advantageously employ a delivery device adapted for holding a shunt or other drainage device, positioning the shunt or drainage device in a preselected position adjacent to the cornea and inserting the shunt or drainage device across the corneal surface to occupy a transcorneal position. In certain embodiments, the delivery device may include an insertion tip adapted for releasably holding the shunt and for positioning the shunt for insertion through the external surface of the cornea, and may further include an inserter slidable from a proximal to a distal position wherein sliding the inserter from the proximal to the distal position dislodges the shunt from the insertion tip and urges it through the external surface of the cornea into the transcorneal position. Advantageously, a pilot hole or other small access wound may be created in the corneal surface or may be extended into or through the corneal stroma before inserting the shunt or drainage device to decrease resistance when the delivery system is used to deliver the device into its preselected transcorneal position. The delivery device according to the present invention may, in certain embodiments, be adapted for indicating to the operator that the shunt has been properly positioned.

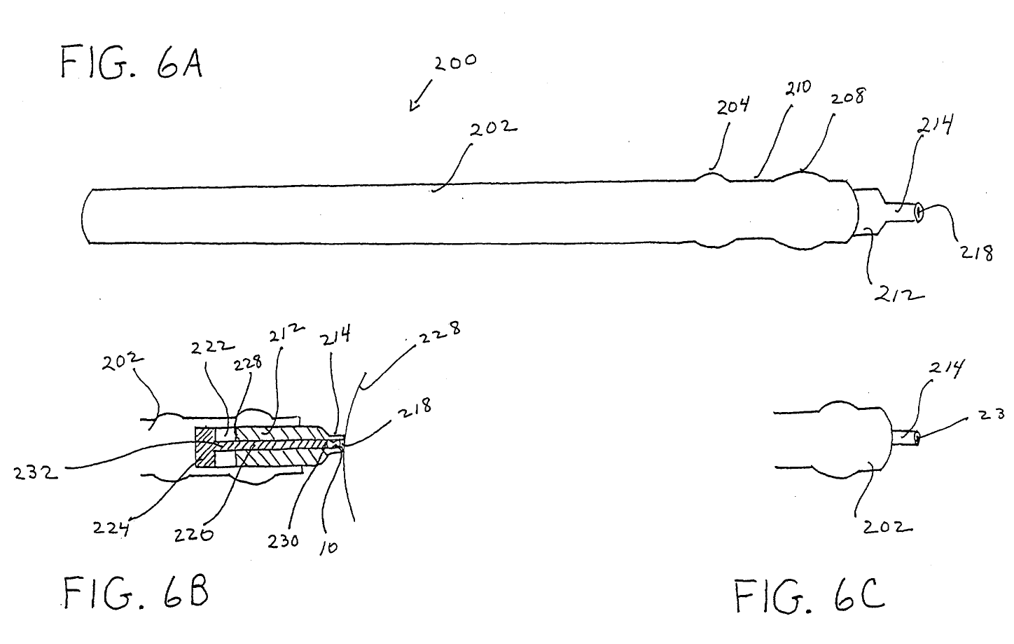

FIG. 6A shows a delivery device 200 suitable for inserting a shunt according to the present invention into a transcorneal position. The delivery device 200 depicted in this figure has an ergonomic design with a proximal elongate shaft 206, a grip area 210, an inserter that includes a slidable tip piece 212, and an insertion tip 214. The shaft 206 and the grip area 210 are formed from a body housing 202, preferably made from a lightweight plastic material. The forward portion of the delivery device 200 includes a hollow distal housing 226 within which the slidable tip piece 212 may be moved anteriorly and posteriorly. The grip area 210 features a proximal protuberance 204 and a distal protuberance 208 between which the delivery device 200 is grasped with a pencil grip, allowing the shaft 206 to rest on the operator's first dorsal web space. The pencil grip is particularly suitable for guiding the insertion tip 214 with precision, although other types of gripping are available for the device 200 at the operator's discretion. At the distal end of the insertion tip 214 is an insertion aperture 218 into which a shunt (not shown) may be placed.

FIG. 6B shows a cross-section of the distal part of a delivery device 200 according to the present invention with the slidable tip piece 212 advanced anteriorly. The slidable tip piece 212 slides coaxially along a fixed plunger 220. FIG. 6B shows the slidable tip piece 212 in a forward position relative to the fixed position of the plunger 220 within the distal housing 226. In this position, a chamber is formed between the distal end 230 of the plunger and the insertion aperture 218 within the insertion top 214 that is dimensionally adapted for releasably holding the shunt 10. In this figure, shunt 10 may be seen positioned within the insertion tip 214 of the slidable tip piece 212, just inside the insertion aperture 218. In this figure, the insertion tip 214 at the distal end of the tip piece 212 is shown in contact with the surface of the cornea 228. So positioned, the anterior face of the shunt 10 is seated approximately flush with the distal insertion tip 214, with the posterior face of the shunt 10 abutting against the distal end 230 of the plunger 220. In this position, furthermore, a posterior chamber 222 is formed posterior to the back end 228 of the slidable tip piece 212 and anterior to the fixed backstop 224. This posterior chamber 222 provides a space into which the slidable tip piece 212 can be pushed by a posteriorly directed force. Such a posteriorly directed force may be produced for the slidable tip piece 212 when the operator advances the delivery device unit 200 forward with its distal insertion tip 214 in contact with the surface 228 of the cornea. The surface 228 of the cornea resists the forward motion of the distal insertion tip 214 and forces the slidable tip piece 212 backwards. The position of the plunger 220, by contrast, is fixed within the delivery device 200. Therefore, as the slidable tip piece 212 is forced relatively backward, the plunger 220 is propelled relatively forward by the continuing advancement of the delivery device 200 in the operator's hand. The plunger 220 and the shunt 10 in contact with the distal end 230 of the plunger 220 continue to move forward so that the shunt is urged past the surface 228 of the cornea into its transcorneal position. Passage of the shunt 10 through the surface 228 of the cornea may be facilitated by providing a small insertion site or pilot hole into which the foot of the shunt (not shown) may enter. The axial length of the sliding chamber 222 may be approximately the same as the length of the shunt 10. This design mitigates against pushing the shunt 10 too far into the eye.

The extent of rearward displacement of the slidable tip piece 212 may be seen in FIG. 6C. In this figure, the insertion tip 214 is visible distal to the distal housing 226, the slidable tip piece 212 having been pushed proximally into the distal housing 226. This figure also shows the distal end 230 of the plunger visible through the insertion aperture 218 of the distal insertion tip 214, indicating that the distal end 230 of the plunger may be approximately flush with the distal end of the insertion tip 214 when the slidable tip piece 212 has been pushed fully backward.

FIG. 6D shows in cross-section the positions of the delivery device structures when the shunt 10 has been pushed through the corneal surface to occupy its transcorneal position across the corneal stroma 232. The slidable tip piece 212 is in its full rearward position, with its back end 228 abutting the backstop 224 of the plunger. The plunger 220 itself is not moveable within the distal housing 226. Instead, forward advancement of the delivery device 200 has pushed the slidable tip piece 212 backward relative to the plunger 220. The shunt 10, remaining in contact with the distal end 230 of the plunger, is urged thereby through the corneal surface 228, advantageously through a pilot hole or incision or insertion site, to occupy its transcorneal position. Further forward directed pressure on the delivery device 200 meets with resistance as the distal insertion tip 214 of the no-longer-displaceable slidable tip piece 212 presses against the corneal surface 228. Encountering this resistance, the operator knows to apply no further pressure.

Other mechanisms may be envisioned to inform the operator that the shunt 10 has been correctly positioned. For example, the posterior chamber 222 may be equipped with notches or tabs (not shown) that mate with correlative structures on the slidable tip piece 212 when the slidable tip piece 212 has been fully displaced rearwardly. The engagement of these mated structures with each other may produce an audible or tactilely perceptible click, informing the operator that full rearward displacement of the slidable tip piece 212 and hence fall forward positioning of the shunt 10 has taken place. The engagement of the mated structures may be permanent, so that the slidable tip piece cannot be returned to its forward position, or the engagement may be releasable by a latch, a button or similar mechanism. Other equivalent structures for signaling the operator about the position of the shunt may be readily envisioned by practitioners in these arts. In certain embodiments, the entire slidable tip piece 212 or the insertion tip 214 may be made from transparent materials, while the plunger may be made from opaque or brightly colored materials. This arrangement may permit the operator easily to perceive the relative positions of these structures with respect to each other. Alternatively, all the distal structures may be made from transparent materials so that the operator can easily visualize the corneal surface through the transparent areas of the delivery device 200.

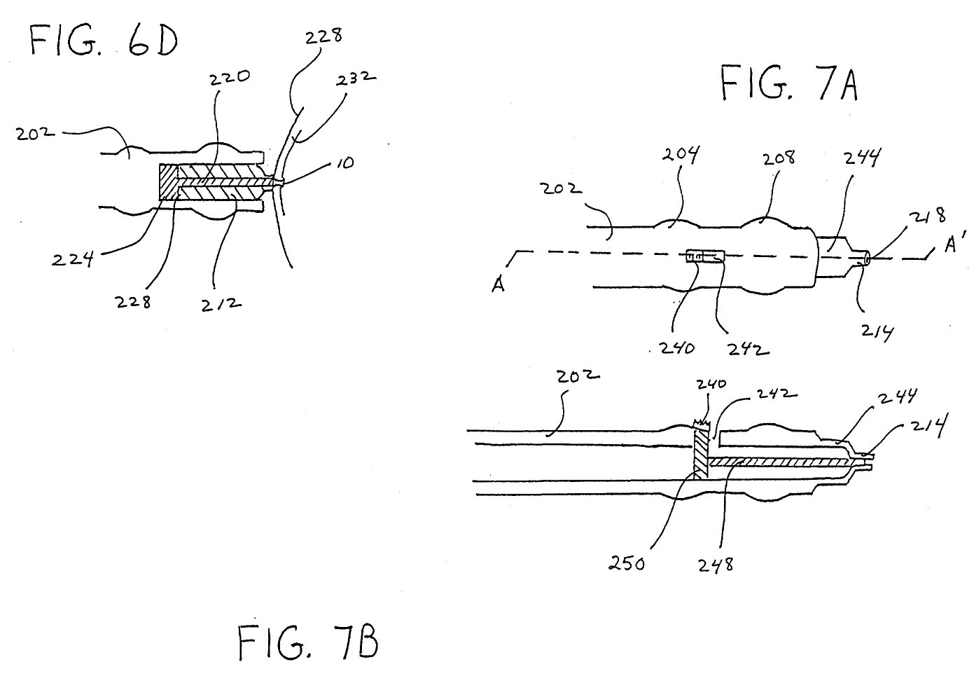

FIG. 7A illustrates yet another embodiment of a delivery device 200 according to the present invention. The outer shape of this embodiment may be similar to the outer shape of the delivery device 200 depicted in FIGS. 6A-D, with, for example, a body housing 202 that extends rearwards to form a shaft (not shown) and a grip area 210 ergonomically formed with a proximal protuberance 204 and a distal protuberance 208. In the depicted embodiment, an insertion aperture 218 is provided at the distalmost part of the insertion tip 214 into which the shunt (not shown) may be releasably inserted. In the depicted embodiment, however, the fixed tip piece 244 and the insertion tip 214 are fixed relative to the delivery device 200. A trigger 240 is provided in proximity to the grip area 210. The trigger 240 is located slidably within a cutout notch 242 through the distal housing 226. The trigger notch 242 permits the forward displacement of the trigger 240 relative to the distal housing 226. As shown in this figure, the trigger is in proximity to the grip area 210, although any other convenient location for the trigger mechanism 240 may be selected. The trigger 240 may have a roughened, corrugated or irregular surface so that it is more maneuverable by an operator.

FIG. 7B shows a longitudinal cross-section of the delivery device 200 taken at line A-A′ of FIG. 7A. While the body housing 202 is shown here as hollow, the body housing 202 proximal to the trigger shaft 250 may be solid or configured in any convenient manner. The distal housing 226, however, is sufficiently hollow to permit axial motion of a slidable plunger 248 therethrough. In the depicted embodiment, the distal housing 226 also bears a cutout trigger notch 242 into which the trigger shaft 250 may be advanced. As shown in this figure, advancement of the trigger shaft 250 forwardly also urges the slidable plunger 248 forward relative to the position of the distal housing 226. This figure shows a chamber 216 present within the insertion tip 214 of the fixed tip piece 224. This chamber 216 is dimensionally adapted for releasably retaining a shunt (not shown) according to the present invention. When the delivery device 200 depicted in this figure is used to insert and position a shunt, the operator may advance the trigger 240 to the forwardmost position of the trigger notch 242, thereby advancing the trigger shaft 250 and its affixed slidable plunger 248 so that the slidable plunger 248 advances into the chamber 216 and displaces the shunt (not shown) therefrom. The insertion tip 214 of the delivery device 200 is adapted for contacting the outer surface of the cornea during shunt delivery. The operator holds the delivery device 200 securely, with its insertion tip 214 in contact with the corneal surface in a preselected position, and the operator then simultaneously advances the trigger 240 forward to insert the shunt through the cornea in the designated area. As has been mentioned previously, a variety of materials may be used for the fabrication of the delivery device 200. In particular, the distal elements of the delivery device may be made of transparent materials. The slidable plunger 248 may also be made of transparent materials, so as to facilitate visualization of the shunt. Alternatively, the insertion tip 214 and/or the fixed tip piece 244 may be made of transparent materials, while the slidable plunger 248 is made of an opaque material that may be brightly colored so that its relative position can be readily visualized.

By referring to the above described drawings, one may appreciate certain methods for decreasing anterior chamber fluid pressure according to the present invention. In one practice of the invention, a shunt is provided to drain aqueous humor, and a delivery device is provided suitable for inserting the shunt. The shunt may be adapted for draining aqueous humor at a preselected rate and further for resisting the incursion of microorganisms. After adequate anesthesia has been provided, a site is selected for insertion of the drainage shunt. A pilot hole may be created that extends across the external surface of the cornea, and that may extend through the corneal stroma and further extend into the anterior chamber. The dimensions of the pilot hole are to be determined by the individual operator, based on surgical judgment and the individual patient's anatomy. A needle, a trocar, a scalpel, or any of the multitude of instruments familiar to ophthalmologic practitioners may be used to form the pilot hole or similar insertion site. The shunt may be inserted by the operator into the delivery device, or the shunt may be pre-inserted in the delivery device during its manufacture. While certain exemplary dimensions for shunt sizes have been disclosed herein, it is understood that a range of shunt sizes may be available to fit the variations in individual anatomy. It is further understood that delivery devices of various sizes may be provided to engage the different sized shunts, or that a single sized delivery device may be suitable for implanting shunts of all different sizes. With the shunt secured in the insertion tip of the delivery device, the operator advances the delivery device toward the external surface of the cornea. When the delivery device reaches the preselected position on the cornea, the shunt is urged into its transcorneal position using the mechanisms of the delivery device for advancing and displacing the shunt. When the shunt has been properly positioned to extend through the cornea, it will be able to drain aqueous humor onto the corneal surface. Proper positioning of the shunt may be evidenced by the presence of a visible droplet of aqueous humor on the head of the implanted device.

It should be understood that such a device may be useful for implantation following those procedures that might be followed by increases in IOP or may be useful as a temporary correction for disorders characterized by increased IOP. In the case of a temporary correction following retina surgery, cataract extractions or other invasive ophthalmic surgeries, the device will be implanted for two hours up to one month, or until IOP has stabilized. In contrast, permanent or otherwise long term implants with the device of the current invention would be used in the case of treating glaucoma in diabetic patients.

It is understood that the specification provided above, with its drawings and descriptions, is only exemplary of the present invention and certain illustrative embodiments. It is further understood that changes and modifications may be made to the various components and structures of the stent and its delivery systems and methods without departing from the scope of the present invention. Rather, the present invention is understood to be defined by the following claims.

We claim:

1. A shunt insertable through a clear cornea of an eye into an anterior chamber thereof, comprising:
a substantially cylindrical body having a channel extending from a proximal end to a distal end of the body for draining aqueous humor from the anterior chamber to an outer surface of the clear cornea;
a head positioned at the distal end of the body for engagement against the outer surface of the clear cornea, the head having an opening therethrough in fluid communication with the channel so as to permit egress of aqueous humor and to minimize ingress of microorganisms;
a foot positioned at the proximal end of the body for engagement against an inner surface of the cornea, the foot having an aperture therethrough in fluid communication with the channel so as to permit inflow of aqueous humor into the channel; and
an elongate filter retainable within the channel for regulating a flow rate of aqueous humor through the channel and for further minimizing the ingress of microorganisms.

2. The shunt of claim 1, wherein at least one of said head and said foot are formed integrally with said body.

3. The shunt of claim 1, wherein at least one of said head, said foot and said body comprise a dehydratable polymer.

4. The shunt of claim 1, wherein said shunt includes a dehydratable polymer whereby dehydration of said shunt reduces the size of said shunt for implantation through a small incision in the cornea and hydration of said shunt provides for said shunt to fit securely in said cornea.

5. The shunt of claim 1, wherein said elongate filter is removable from the channel.

6. The shunt of claim 1, wherein the body comprises a hydrogel.

7. The shunt of claim 6, wherein the hydrogel is covalently crosslinked and is based on a methacrylic acid derivative.

8. The shunt of claim 1, wherein at least one of an external surface of the head and an external surface of the foot are configured to minimize cellular adhesion.

9. The shunt of claim 1, wherein an external surface of the body is configured to encourage tissue adhesion.

10. The shunt of claim 1, wherein the foot is tapered to facilitate insertion of the corneal shunt through the cornea.

11. The shunt of claim 1, wherein the foot is dimensionally alterable from a first configuration to a second configuration to facilitate insertion of the corneal shunt through the cornea.

12. The shunt of claim 1, wherein the elongate filter is retainable within the channel by impaction.

13. The shunt of claim 1, wherein the elongate filter is retainable proximally within the channel.

14. An implant for transcorneal placement to drain an anterior chamber of an eye, comprising:
a head adapted for resting upon an external aspect of a cornea having a slit to permit egress of aqueous humor while resisting ingress of microorganisms and having an exterior surface resistant to cell adhesion;
a foot adapted for insertion across the cornea into the anterior chamber and further adapted to abut an internal aspect of the cornea atraumatically, having an aperture to permit outflow of aqueous humor therethrough;
a tubular conduit between the foot and the head having an interior channel in fluid communication with the aperture and the slit and having an external surface resistant to cell adhesion; and
an elongate filter dimensionally adapted for retention within the interior channel and provided with filtration pores to regulate rate of outflow of aqueous humor and to restrict the incursion of microorganisms.

15. A system for reducing intraocular pressure, comprising:
a transcorneal shunt to drain aqueous humor from an anterior chamber to an external surface of a cornea; and
a delivery device for implanting the shunt transcorneally, comprising
an insertion tip dimensionally adapted for releasably holding the shunt and for positioning the shunt for insertion through the external surface, and
an inserter slidable from a first position to a second position, wherein sliding said inserter from the first position to the second position dislodges the shunt from the insertion tip and urges the shunt through the external surface into a transcorneal position;
wherein the drainage of the aqueous humor from the anterior chamber to the external surface of the cornea by the shunt reduces intraocular pressure.

16. The system of claim 15, wherein the transcorneal shunt has an elongate tubular body, a head, a foot, and a filter, said body having a channel extending from one end to an opposite end for draining aqueous fluid therethrough, said head being positioned at the one end of the body for engagement against the external surface and having a slit in communication with the channel to permit egress of aqueous humor onto the external surface and to restrict ingress of microorganisms, said foot being positioned at the opposite end of the body for engagement against an internal surface of the cornea and having an aperture in communication with the channel to permit introduction of aqueous humor therein, and a filter retainable within the channel for regulating a flow rate of aqueous humor therethrough and for further restricting ingress of microorganisms.

17. The system of claim 15, wherein the inserter comprises a slidable tip piece movable from anterior to posterior, and wherein the delivery device further comprises a fixed plunger coaxial with the slidable tip piece.

18. The system of claim 15, wherein the delivery system further comprises a fixed distal tip piece, and wherein the inserter comprises a slidable plunger coaxial with the fixed distal tip piece and movable from posterior to anterior.

19. A method for decreasing anterior chamber fluid pressure, comprising:
providing a shunt to drain aqueous humor from an anterior chamber to an external surface of the cornea;
providing a delivery device having a tip dimensionally adapted for releasably retaining the shunt and for positioning the shunt for insertion through the external surface and having an inserter that displaces the shunt from the tip and urges the shunt through the external surface into a transcorneal position; and
employing the delivery device to insert the shunt across the cornea into the transcorneal position, whereby aqueous humor can flow from the anterior chamber to the external surface, thereby decreasing anterior chamber fluid pressure.

20. The method of claim 19, wherein the shunt has an elongate tubular body, a head, a foot, and a filter, said body having a channel extending from one end to an opposite end for draining aqueous fluid therethrough, said foot being positioned at the one end of the body for engagement against an internal surface of the cornea and having an aperture in communication with the channel to permit introduction of aqueous humor therein, said head being positioned at the opposite end of the body, being adapted for abutting the external surface and having a slit in communication with the channel to permit egress of aqueous humor onto the external surface, and said filter retainable within the channel for regulating a flow rate of aqueous humor therethrough and for further restricting ingress of microorganisms.

21. The method of claim 19, further comprising creating a pilot hole through the external surface to permit the insertion of the shunt therethrough.

22. The method of claim 19, further comprising removing the shunt after a preselected period of time.

23. The method of claim 22, wherein said preselected period of time is less than one month following surgery.

24. The method of claim 22, wherein said preselected period of time is at least one month.

25. The method of claim 22, wherein said preselected period of time is at least two hours following surgery.

26. A transcorneal implant spanning a cornea between a tear film on an external aspect of the cornea and an anterior chamber of an eye, comprising:
a head protruding from the cornea and having an outer surface in contact with the tear film and in contact at least intermittently with an eyelid, said outer surface being wettable with tears, highly hydrated and resistant to cell adherence;
a body comprising a hydrogel and having an external surface contacting stromal tissue of the cornea, said external surface being less hydrated than said outer surface of said head and facilitating cell adherence; and
a foot protruding into the anterior chamber.

27. The implant of claim 26, wherein the body is penetrated by an internal cavity having an internal surface.

28. The implant of claim 27, wherein the internal cavity includes a channel connecting the anterior chamber with the tear film.

29. The implant of claim 28, wherein the channel contains a filter that obstructs passage of microorganisms.

30. A method for manufacturing a corneal implant comprising casting a mixture comprising HEMA, methacrylic acid, dimethacrylate crosslinker, and a free radical initiator into a single part silicone mold with a cavity formed by imprinting with a die shaped in a preselected shape.

31. A method for manufacturing a corneal implant comprising machining a shunt and applying a tissue integration layer to an outer surface of the shunt, said tissue integration layer comprising a curable composition comprising a copolymer of HEMA with alkylmethacrylate, monomer HEMA, a dimethacrylate crosslinker, a free radical initiator and a volatile solvent.
